(19) United States
(12) Patent Application Publication
(10) Pub. No.: US 2021/0401324 A1
(43) Pub. Date: Dec. 30, 2021

(54) METHOD FOR RECOGNIZING A MOTION PATTERN OF A LIMB

(71) Applicant: The Chinese University of Hong Kong, Hong Kong (CN)

(72) Inventors: Wei-Hsin Liao, Hong Kong (CN); Fei Gao, Neijiang (CN); Gao-Yu Liu, Guilin (CN)

(21) Appl. No.: 16/949,581

(22) Filed: Nov. 4, 2020

(30) Foreign Application Priority Data
Jun. 28, 2020 (CN) 202010598429.0

(51) Int. Cl. A61B 5/11; A61B 5/00; A61B 5/103; A61B 5/107; G06K 9/00; G06K 9/62; G06T 7/20

(52) U.S. Cl. CPC A61B 5/112; A61B 5/7267; A61B 5/1038; A61B 5/1071; G06T 2207/30241; G06K 9/00348; G06K 9/6269; G06T 7/20; A61B 5/6829

(57) ABSTRACT

The present application relates to a method for recognizing motion pattern of human lower limb and prostheses, orthoses, or exoskeletons thereof. The method may comprise collecting motion data; inputting the collected motion data and corresponding limb motion patterns into a classifier or pattern recognizer to train the classifier or the pattern recognizer; and inputting the motion data of the limb obtained in real time by the sensor into the trained classifier or the trained pattern recognizer to recognize a motion pattern of the limb.

### CROSS-REFERENCE TO RELATED APPLICATION

This application claims benefit of priority to Chinese Patent Application No. 202010598429.0 filed on Jun. 28, 2020 before the China National Intellectual Property Administration, the entire disclosure of which is incorporated herein by reference in its entity.

### TECHNICAL FIELD

The present application relates to a technical field of recognizing motion pattern of a limb, for example, recognizing motion pattern of lower limbs and prostheses, orthoses or exoskeletons of a human body.

### BACKGROUND

With the progress of science and technology and the improvement of human living standard, the research and development of rehabilitation medical equipment for people is gradually and increasingly concerned by the society and government. Recently, there has been a significant increase in the demand for human power aids or medical rehabilitation training equipment in stroke hemiplegia, impaired motion function of lower limb or disabled persons. The lower limb rehabilitation training equipment may help stroke hemiplegic patients or motion-function-impaired patients to regain the walking ability and thus improve the quality of life. In addition, the lower limb rehabilitation training equipment may also help to restore the motion function to injured muscles or joints and reduce or eliminate permanent physical impairment. In addition, some researchers are working on the development of various intelligent human power aids for soldiers or heavy-duty carriers, hoping to greatly improve the weight-bearing capacity for the users while reducing their walking or work burden.

It has been noted that in different motion patterns, such as upslope, downslope, upstairs or downstairs, the function performed by each joint of the lower limb of human body and the corresponding biomechanical characteristics vary considerably. Therefore, in order to achieve the desired function more accurately, the lower limb auxiliary device firstly should be able to accurately recognize the motion pattern of the user (wearer), and then control a driver to generate a preset auxiliary torque according to the corresponding motion pattern, thereby assisting the wearer to perform the desired action more easily.

In order to realize the motion pattern recognition functions of the human lower limb, the lower limb orthopedic device and the exoskeleton as described above, a variety of implementation methods have been proposed. Some researchers have proposed to detect the motion pattern of a wearer who wears the lower limb auxiliary device in real time by extracting and analyzing the electromyographic (EMGs) or electroencephalographic (EEG) signals of is the wearer. However, the recognition accuracy of such methods is greatly reduced due to the fact that muscles are prone to fatigue and body sweating during long-term exercise. Furthermore, the EEG signals have a plurality of dimensions and the computation load for the EEG signals is heavy, and thus it is difficult to realize real-time pattern recognition on mobile devices at present. In addition, it has been proposed to analyze the motion pattern of the wearer based on the pressure signal of a foot of the wearer.

However, it should be noted that when the ground surface is uneven or the walking speed of the wearer is changed, the performance of such pattern recognition method will be greatly degraded, and therefore, it is difficult to be widely used in real scenarios. According to another prior art, it has been proposed to use dynamic information obtained by an inertial measurement unit fixed to the tendon side or embedded in the prosthesis for motion intention recognition of the prosthesis. However, considering that the dynamic information in the sensor reference coordinate system, which is obtained during the motion, is related to the motion speed of the wearer, it would be difficult to popularize this method in practical application.

### SUMMARY

The disclosures of the present application propose methods for recognizing motion pattern of limbs and prostheses, orthoses or exoskeletons of the human body.

In one aspect of the present application, there is provided a method for recognizing a motion pattern of a limb, and the method may comprise: collecting, by a sensor, motion data of a limb extremity end of a subject during a swing stage of the extremity end in different motion modes; training a classifier or a pattern recognizer by inputting the collected motion data and corresponding limb motion patterns into the classifier or the pattern recognizer to train; and recognizing the motion pattern of the limb by inputting the motion data of the limb, which is obtained in real time by the sensor, into the trained classifier or the trained pattern recognizer.

According to exemplary embodiments of the present application, wherein the limb may for example comprise a lower limb, lower limb prosthesis, a lower limb orthosis, a lower limb exoskeleton of a human body or the like, and the motion patterns may for example comprise upslope, downslope, upstairs, downstairs, walking on flat ground, turning and the like.

According to an exemplary embodiment of the present application, the motion data may comprise one or more of an absolute motion trajectory to ground, an absolute velocity to ground, and an absolute acceleration to ground of the limb extremity end during the swing stage in the different motion modes.

According to exemplary embodiments of the present application, the sensor may comprise an inertial measurement unit fixed to the limb extremity end. The method may further comprise: obtaining one or more of the absolute motion trajectory to ground, the absolute velocity to ground, and the absolute acceleration to ground through a coordinate transformation and an integration (e.g., first order integration or second order integration) of angular velocity and acceleration data of the inertial measurement unit, which are obtained in a sensor coordinate system.

According to exemplary embodiments of the present application, the method may further comprise resetting, when the subject is in a standing stage, a transformation matrix for the coordinate transformation, the absolute velocity to ground, and an absolute motion displacement to ground, to eliminate or reduce a cumulative drift or cumulative error of the inertial measurement unit.

According to exemplary embodiments of the present application, wherein the method may further comprise detecting the standing stage of the subject by the inertial measurement unit fixed at the limb extremity end or a load cell mounted on a foot of the subject.

According to exemplary embodiments of the present application, the is collecting motion data may comprise extracting the absolute motion trajectory to ground of the limb extremity end in a sagittal plane, or deriving terrain slopes corresponding to the different motion patterns from the absolute motion trajectory to ground in the sagittal plane to recognize the motion pattern being performed.

According to exemplary embodiments of the present application, the method may further comprise triggering, based on a trigger boundary condition, the trained classifier or the trained pattern recognizer to recognize the motion pattern performed by the subject before a foot of the subject touching ground. The motion pattern of the subject can be recognized in response to the trigger boundary condition being satisfied.

According to exemplary embodiments of the present application, the trigger boundary condition may for example comprise an elliptical boundary condition, a circular boundary condition, or a rectangular boundary condition. The motion pattern of the subject can be recognized when the absolute motion trajectory to ground of the limb extremity end passes through the trigger boundary condition.

According to exemplary embodiments of the present application, the trigger boundary condition may for example further comprise one or more of a time threshold trigger, an absolute displacement to ground trigger in a forward direction or a direction vertical to ground, an absolute velocity to ground trigger, or an absolute acceleration to ground trigger.

According to exemplary embodiments of the present application, the trigger boundary condition may comprise one or more of the angular velocity or acceleration signals of the inertial measurement unit in the sensor coordinate system satisfy a preset trigger condition.

According to exemplary embodiments of the present application, the method may further comprise detecting, based on a time window, the motion pattern of the subject in real time to recognize the motion pattern performed by the subject before a foot of the subject touches the ground. The motion pattern of the subject can be recognized in response to one or more of the absolute velocity to ground, the absolute acceleration to ground, or the absolute motion trajectory to ground matching, within the time window, a corresponding data of a particular motion pattern.

According to exemplary embodiments of the present application, wherein collecting motion data of the limb extremity end of the subject during the swing stage in different motion modes may comprise calculating a rotation angle or angular velocity of the limb extremity end relative to an initial sagittal plane or an initial coronal plane of the subject to recognize turning activity of the subject.

According to exemplary embodiments of the present application, the method may further comprise obtaining the rotation angle or angular velocity of the limb extremity end relative to the initial sagittal plane or the initial coronal plane of the subject by converting output data of the inertial measurement unit fixed to the limb extremity end, or recognizing the turning activity of the subject by detecting the rotation angle or angular velocity of other parts (e.g., head, upper torso, arms, lower thighs, lower legs, feet, etc.) of the body of the subject relative to the initial sagittal plane or the initial coronal plane of the subject.

According to exemplary embodiments of the present application, the classifier or the pattern recognizer for motion pattern recognition of the limb may comprise, for example, a linear discriminant analyzer, a quadratic discriminant analyzer, a support vector machine, a neural network, or the like, but the present application is not limited thereto.

According to exemplary embodiments of the present application, the sensor may further comprise an inertial measurement unit-combined laser displacement sensor mounted on lower legs, thighs, waists, head or other portion of the subject. The inertial measurement unit-combined laser displacement sensor may be configured to measure one or more of the absolute motion trajectory to ground, the absolute velocity to ground, or the acceleration to ground, or measure directly topographic characteristics in the different motion patterns.

According to exemplary embodiments of the present application, the sensor may further comprise an inertial measurement unit-combined depth camera mounted to lower legs, thighs, waists, head or other portion of the subject. The inertial measurement unit-combined depth camera may be configured to measure one or more of the absolute motion trajectory to ground, the absolute velocity to ground, or the acceleration to ground, or measure directly topographic characteristics in the different motion patterns.

According to exemplary embodiments of the present application, the sensor may further comprise an infrared capture system mounted in an ambient environment of the subject, and an infrared capture marker point is mounted at the limb extremity end of the subject. The method may further comprise analyzing one or more of the absolute motion trajectory to ground, the absolute velocity to ground, and the absolute acceleration to ground of the infrared capture marker point to recognize the motion pattern of the subject.

According to exemplary embodiments of the present application, the method may further comprise recognizing the different motion patterns by combining one or more of the absolute motion trajectory to ground, the absolute velocity to ground, and the absolute acceleration to ground with a foot pressure distribution of the subject, a rotation angle of a lower limb knee joint or ankle joint, an electromyographic signal or an electroencephalographic signal (EEG) of the subject.

According to exemplary embodiments of the present application, the method may further comprise recognizing the different motion patterns by combining one or more of the absolute motion trajectory to ground, the absolute velocity to ground, and the absolute acceleration to ground with an angular velocity or an acceleration in a sensor coordinate system measured by an inertial measurement unit fixed at the limb extremity end.

In another aspect of the present application, there is provided a non-transitory machine-readable medium having instructions stored therein, which when executed by a processor, cause the processor to perform operations of collecting, by a sensor, motion data of a limb extremity end of a subject during a swing stage of the extremity end in different motion modes; inputting the collected motion data and corresponding limb motion patterns into a classifier or a pattern recognizer to train the classifier or the pattern recognizer; and inputting the motion data of the limb, which is obtained in real time by the sensor, into the trained classifier or the trained pattern recognizer to perform motion pattern recognition of the limb.

In another aspect of the present application, there is provided a data processing system comprising a processor and a memory, wherein the memory is coupled to the processor to store instructions which, when executed by the processor, cause the processor to perform operations of collecting, by a sensor, motion data of a limb extremity end of a subject during a swing stage in different motion modes; inputting the collected motion data and corresponding limb motion patterns into a classifier or a pattern recognizer to train the classifier or the pattern recognizer; and inputting the motion data of the limb, which is obtained in real time by the sensor, into the trained classifier or the trained pattern recognizer to perform motion pattern recognition of the limb.

Other features and aspects of the present application will be apparent from the following detailed description, the drawings, and the claims.

### BRIEF DESCRIPTION OF THE DRAWINGS

The principles of the inventive concept are illustrated below by describing non-limiting embodiments of the present disclosure in conjunction with the accompanying drawings. It should be understood that the drawings are intended to illustrate, rather than limit the exemplary embodiments of the present disclosure. The accompanying drawings are included to provide a further understanding of the general concept of the present disclosure, and are incorporated in the specification to constitute a part thereof. The same reference numerals in the drawings denote the same features. In the accompanying drawings:

FIG. 1 shows a schematic diagram of an absolute motion trajectory to ground of a lower limb extremity end of human body when traveling on terrains having different slopes according to an embodiment of the present application;

FIG. 2 shows a flowchart of a method for recognizing a motion pattern of a limb according to an embodiment of the present application;

FIG. 3 shows a schematic diagram of a sensor mounted to the lower limb extremity end of human body for detecting the absolute motion trajectory of the lower limb extremity end relative to the ground during walking according to an embodiment of the present application;

FIG. 4 shows a schematic diagram of a motion capture system mounted in a human surrounding environment for measuring an absolute motion trajectory of a marker point mounted at the lower limb extremity end of human body relative to the ground during walking according to an embodiment of the present application;

FIG. 5 shows a schematic diagram of detecting a standing stage and a swinging stage in a walking process of human body by using an acceleration signal output from an inertial measurement unit mounted on the lower limb extremity end of human body, while resetting a displacement of the lower limb extremity end in a forward direction and a direction vertical to ground during the standing stage according to an embodiment of the present application;

FIG. 6 shows a schematic diagram of an elliptical boundary condition for triggering motion pattern recognition decision for human lower limb according to an embodiment of the present application, wherein a motion pattern decision will be triggered when the obtained absolute motion trajectory to ground passes through the elliptical boundary condition;

FIG. 7 shows a schematic diagram for classifying and recognizing the movement pattern of human lower limb based on a series of threshold values with a ground slope obtained by derivation according to an embodiment of the present application;

FIG. 8 shows a schematic diagram for recognizing a turning motion of a human body based on a rotation angle of the lower limb extremity end of human body relative to an initial sagittal plane of the human body according to an embodiment of the present application; and

FIG. 9 shows a simplified block diagram of an information handling system (or computing system) for the above method of recognizing the motion pattern of a limb, according to embodiments of the present disclosure.

### DETAILED DESCRIPTION

For a better understanding of the present disclosure, various aspects of the present disclosure will be described in more detail with reference to the exemplary embodiments illustrated in the accompanying drawings. It should be understood that the detailed description is merely an illustration of the exemplary embodiments of the present disclosure rather than a limitation to the scope of the present disclosure in any way. Throughout the specification, like reference numerals refer to like elements. The expression “and/or” includes any and all combinations of one or more of the associated listed items.

In the accompanying drawings, the thicknesses, sizes and shapes of the components have been slightly exaggerated for the convenience of explanation. The accompanying drawings are merely illustrative and not strictly drawn to scale.

It should be understood that the terms “comprising”, “including”, “having” and variants thereof, when used in the specification, specify the presence of stated features, elements, components and/or steps, but do not exclude the presence or addition of one or more other features, elements, components, steps and/or combinations thereof. In addition, expressions, such as “at least one of”, when preceding a list of listed features, modify the entire list of features rather than an individual element in the list. Further, the use of “may”, when describing the embodiments of the present disclosure, relates to “one or more embodiments of the present disclosure”. Also, the term “exemplary” is intended to refer to an example or illustration of the embodiment.

Unless otherwise defined, all terms (including technical and scientific terms) used herein have the same meaning as commonly understood by those skilled in the art to which the present disclosure belongs. It should be further understood that terms, such as those defined in commonly used dictionaries, should be interpreted as having a meaning that is consistent with their meaning in the context of the relevant art and will not be interpreted in an idealized or overly formal sense unless explicitly so defined herein.

The various aspects of the present disclosure are described in more detail below with reference to the accompanying drawings and in conjunction with specific embodiments, but the embodiments of the present disclosure are not limited thereto.

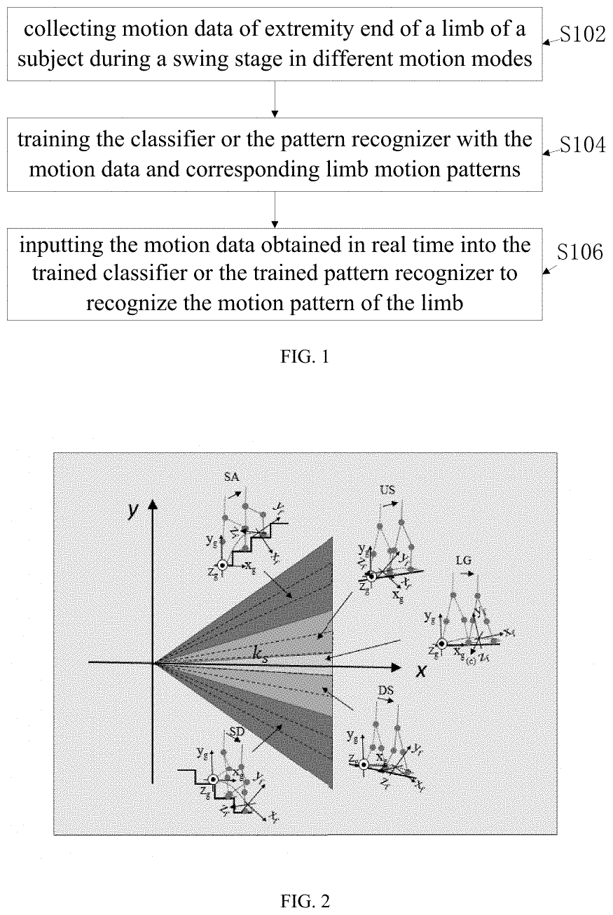

FIG. 1 shows a schematic diagram of an absolute motion trajectory to ground of a lower limb extremity end of human body when traveling on terrains having different slopes according to an embodiment of the present application.

As shown in FIG. 1, a living environment surrounding persons is constructed according to slopes of ground. For example, when the slope of the ground is close to zero, the ground is flat; when the slope of the ground is small, the ground may be constructed as a slope; when the slope of ground is large, the ground construction may be stairway in consideration of ergonomics and safety. For example, when an inclination angle of the ground is in a range of 7 to 15 degree, the ground environment is typically constructed as a ramp walkway. However, when the inclination angle of the ground is in a range of 30 to 35 degree, the ground environment will be constructed as a stair walkway. Based on this, the types of terrain can be classified or recognized according to the slope of the ground, thereby recognizing the motion patterns performed by human body under the terrain. In addition, during walking, the lower limb extremity end of human body (e.g., the foot) is usually moved along the ground to reduce the power consumption of the human body for moving the lower limb during walking, while obtaining the necessary off-ground clearance to prevent from falling down due to a collision with the ground. In view of this, an absolute motion trajectory to ground, an absolute velocity to ground, and an absolute acceleration to ground of the lower limb extremity end of human body can partially reflect the characteristics of the corresponding terrain, and can be used to distinguish the motion pattern performed by human body. In addition, the geometrical characteristics of the ground can be directly obtained by means of a sensor installed on the human body or in the surrounding environment, so that the motion pattern performed by human body can be distinguished or identified.

The present invention proposes to derive the slope of the ground of the corresponding terrain based on the absolute motion trajectory to ground of the lower limb extremity end of human body, thereby distinguishing or predicting the performed motion pattern.

The method according to an embodiment of the present application is a pattern recognition or classification method based on parameter training.

FIG. 2 shows a flowchart of a method for recognizing a motion pattern of a limb according to an embodiment of the present application. As shown in FIG. 2, in step S102, training data for a classifier or a pattern recognizer can be collected through a large number of experimental tests; In step S104, the motion data and the corresponding limb motion pattern may be input to the classifier or the pattern recognizer to train the classifier or the pattern recognizer; and in step S106, motion data of the limb obtained in real time by the sensor may be input to the trained classifier or the trained pattern recognizer to perform motion pattern recognition of the limb. The above steps are further described below.

### Step S102: Collecting Training Data

During the data collection process, a certain number of subjects are required to repeat several common motion patterns in daily life according to an experimental protocol, for example, as shown in FIG. 1, including: upslope US, downslope DS, upstairs SA, downstairs SD, flat ground walking LG, turning, etc., to obtain sufficient training data.

In this step, one or more of the absolute motion trajectory to ground, the absolute velocity to ground, or the absolute acceleration to ground of the lower limb extremity end of human body in various daily motion patterns can be measured directly or indirectly by means of a sensor installed on the human body or in the environment surrounding the human body, and then the measured data is input to a pattern recognizer or a classifier so as to realize the detection of the motion pattern (e.g., upslope, downslope, upstairs, downstairs, and flat ground walking) of human body.

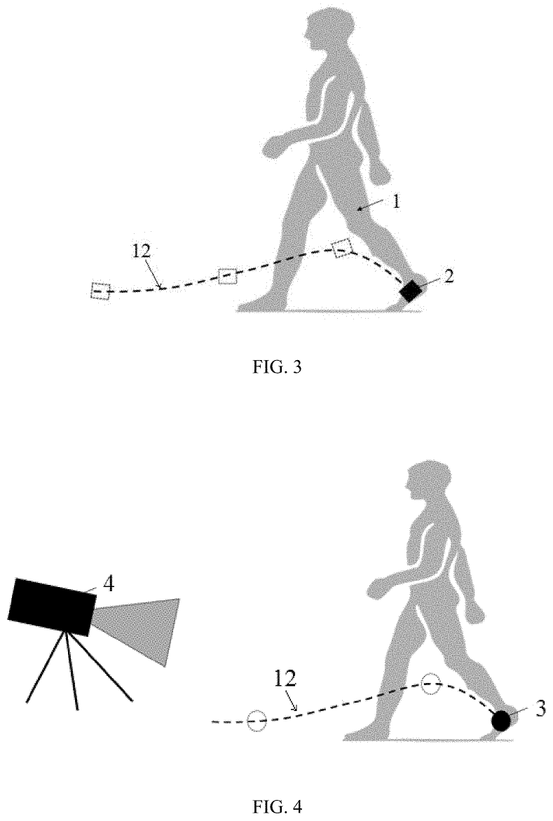

FIG. 3 schematically shows a schematic view for detecting the absolute motion trajectory to ground 12 of the lower limb extremity end of 1 directly or indirectly by means of a sensor 2 mounted on the lower limb extremity end of 1 of human body. The sensor 2 may be, for example, an inertial measurement unit, an inertial measurement unit-combined laser displacement sensor, or an inertial measurement unit-combined depth camera.

In an exemplary embodiment, the above-described sensor 2 for measuring the absolute motion trajectory to ground of the lower limb extremity end of human body may be, for example, an inertial measurement unit mounted at the lower limb extremity end of human body, such as any position of the ground-proximal end of the lower leg, the heel, the toe, or the foot, or mounted at a corresponding position of a lower limb, prosthesis, orthosis, or an exoskeleton. The inertial measurement unit can obtain the is angular velocity and the acceleration in the sensor coordinate system, and conversion matrixes, attitude angle of the sensor, the absolute motion trajectory to ground at the lower limb extremity end, the absolute velocity to ground, and the absolute acceleration to ground can be obtained by performing a coordinate transformation, a first order integration, and a second order integration.

In another exemplary embodiment, the above-described sensor 2 for measuring the absolute motion trajectory to ground of the lower limb extremity end of human body may be, for example, an inertial measurement unit-combined laser displacement sensor mounted at the lower limb extremity end of human body or a corresponding position on a prosthesis, an orthosis or an exoskeleton for the lower limb. The inertial measurement unit-combined laser displacement sensor may be mounted at other portions of the human body, such as head, waists, thighs, or lower legs. In addition, the inertial measurement unit-combined laser displacement sensor may be used to directly measure terrain features, thereby recognizing the motion pattern performed by human body.

In yet another exemplary embodiment, the above-described sensor for measuring the absolute motion trajectory to ground of the lower limb extremity end of human body may be a depth camera mounted at the lower limb extremity end of human body or a corresponding position on a prosthesis, an orthosis or an exoskeleton for the lower limb. The depth camera may be mounted to other portions of the human body, such as head, waists, thighs, or lower legs. In addition, the depth camera may be used to directly measure terrain features, thereby recognizing the motion pattern performed by human body.

It should be noted that when the sensor 2 is an inertial measurement unit-combined laser displacement sensor or an inertial measurement unit-combined depth camera, the sensor 2 may, for example, be mounted on other body parts such as a lower leg, a thigh, a waist or a head for measuring ground features, thereby recognizing or classifying terrain types and recognizing the motion pattern of the lower limb of human body.

In addition, one or more of the measured absolute motion trajectory to ground, the absolute velocity to ground, and the absolute acceleration to ground of the lower limb extremity end may be combined with one or more of the acceleration and the angular velocity in the sensor coordinate system obtained by the inertial measurement unit mounted at the lower limb extremity end to recognize the motion pattern for human body.

In addition, although not shown in the drawings, one or more of the measured absolute motion trajectory to ground, the absolute velocity to ground, and the absolute acceleration to ground of the lower limb extremity end may be combined with signal such as human foot pressure distribution signal, EMG signal, EEG signal, rotation angle of each joint of the lower limb or the like to improve the recognition accuracy of the existing motion pattern recognizer.

In yet another exemplary embodiment, the above-described sensor for measuring the absolute motion trajectory to ground of the lower limb extremity end of human body may be a motion capture system installed in the environment surrounding the human body. In this case, it is necessary to dispose capture marker points at the lower limb extremity end of human body. In addition, it is also possible to dispose capture marker points at other parts of the lower limb of human body, such as a knee joint, an ankle joint, a lower leg, a thigh, or the like. The dynamic capture system may obtain the absolute motion trajectory to ground, the absolute velocity to ground, and the absolute acceleration to ground of the capture marker points for recognizing the motion pattern of human body.

FIG. 4 schematically illustrates a schematic diagram of a motion capture system mounted in an environment surrounding the human body, which can be used to detect a motion trajectory of the lower limb extremity end of human body. In the case that the motion capture system 4 is mounted in the environment surrounding the human body, it is necessary to dispose the capture marker points 3 at the lower limb extremity end of human body. It should be noted that the capture marker points may also be disposed at other parts of the lower limb of human body (e.g., ankle joint, knee joint, etc.). The corresponding motion pattern may be recognized by analyzing signals such as the absolute motion trajectory to ground, the absolute velocity to ground, and the absolute acceleration to ground of the capture marker points 3.

Step S104: Training the Pattern Recognizer or Classifier

Referring again to FIG. 1, after the motion data of the extremity end of the subject in the swing stage of different motion patterns are collected, the obtained training data may be input to the pattern recognizer or the classifier to repeatedly train the pattern recognizer or the classifier with a plurality of times in step S104 to meet the required accuracy requirements. Thus, the parameter setting in the pattern recognizer or the classifier is completed.

In an exemplary embodiment, the collected motion data of the extremity end of the subject in the swing stage of different motion patterns can be classified or recognized using common pattern recognition methods. For example, the motion data may be classified or recognized by a linear discriminant analyzer, a secondary discriminant analyzer, a support vector machine, or a neural network. The motion data may include, for example, an absolute motion trajectory to ground, an absolute velocity to ground, an absolute acceleration to ground, and the like. It is also possible to perform data processing to the above motion data (e.g., the absolute motion trajectory to ground) to obtain a corresponding slope of the ground, thereby recognizing the motion pattern performed by the lower limb of human body.

### Step S106: Recognizing the Motion Pattern of the Limb

Referring again to FIG. 1, after the parameter setting in the pattern recognizer or the classifier is completed, as shown in step S106, the data used in the training can be collected in real time by the sensor, and the trained pattern recognizer or the trained classifier can detect the motion mode according to the input signals.

In order to eliminate the drift or the accumulated error that may occur in the later data processing of the output signal of the inertial measurement unit, it is necessary to correct and reset the conversion matrix, the absolute velocity to ground, the absolute motion trajectory to ground, and the like at the standing stage of the lower limb of human body. The standing stage may be detected by the output signal of the inertial measurement unit, or by the pressure sensor of the foot or the axial force sensor of the orthopedic, prosthetic limb and exoskeleton.

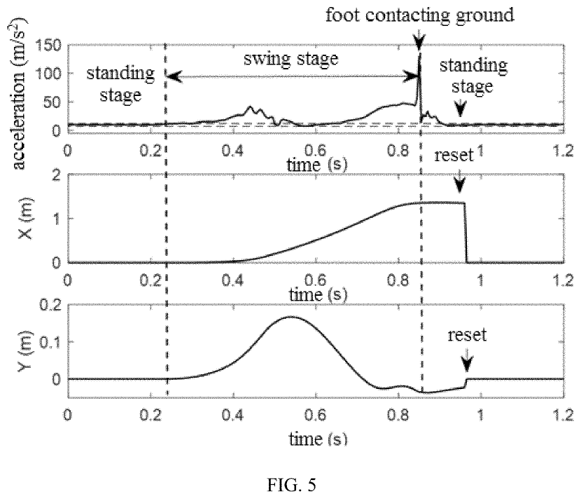

FIG. 5 shows a schematic diagram of detecting a standing stage and a swinging stage in a walking process of human body by using an acceleration signal output from an inertial measurement unit mounted on extremity end of human lower limb, while resetting a displacement of the extremity end of lower limb in a forward direction and a direction vertical to ground during the swinging stage according to an embodiment of the present application. As shown in FIG. 5, when the sensor 2 is an inertial measurement unit, the walking state of the human body can be detected from the acceleration output signal of the inertial measurement unit according to the following equation

$$state = \{ \begin{matrix}if ‖ {a}_{f} - {a}_{g} ‖ < {ξ}_{f} & standing stage \\ otherwise & swing state\end{matrix} \qquad ( 1 )$$

Wherein, α_{f} represents the acceleration of the inertial measurement unit, α_{g} represents the acceleration of gravity, and ξ_{f} is a predetermined threshold value.

When the absolute value of the acceleration signal of the inertial measurement unit obtained by measurement is close to the gravity acceleration for a period of time, the lower limb of human body is considered to be in the standing stage. To eliminate the accumulated error of the inertial measurement unit, the conversion matrix of the sensor, the absolute displacement to ground, and the absolute velocity to ground are reset. When the absolute value of the acceleration signal obtained by the measurement is greater than the gravity acceleration, the lower limb of human body is in the swing state, and the absolute motion displacement to ground and the absolute velocity to ground of the lower limb extremity end, and the conversion matrix are updated.

As shown in FIG. 5, during a first standing stage S1, the acceleration α_{f} of the inertial measurement unit is close to the gravity acceleration α_{g}, and the absolute value of the subsequently measured acceleration of the inertial measurement unit is greater than the gravity acceleration α_{g}, thereby determining that the lower limb of human body is in a swing stage S2. After the swing stage S2, when the measured acceleration α_{f} of the inertial measurement unit is close to the gravity acceleration α_{g}, it is determined that the lower limb of human body is in a second standing stage S3, and the conversion matrix of the sensor (e.g., the inertial measurement unit), the absolute displacement to ground, and the absolute velocity to ground are reset at the second standing stage S3.

In order to be able to recognize the motion pattern of the lower limb, prosthesis, orthosis or exoskeleton before the next foot contacting the ground, thereby enabling the lower limb, prosthesis, orthosis or exoskeleton to complete the required preparation during the swing stage, a predetermined triggering condition may be used to trigger the pattern recognition decision of the classifier or the pattern recognizer. For example, during downstairs, the human ankle needs to extend during the swing stage. In order to reduce the impact force by bending the ankle joint to cushion the collision during the foot contacting the ground, a triggering boundary condition for pattern recognition may be employed. In addition, a data window may be used to match the input data with the corresponding data in a specific pattern in real time to realize real-time detection of the motion pattern of the lower limb.

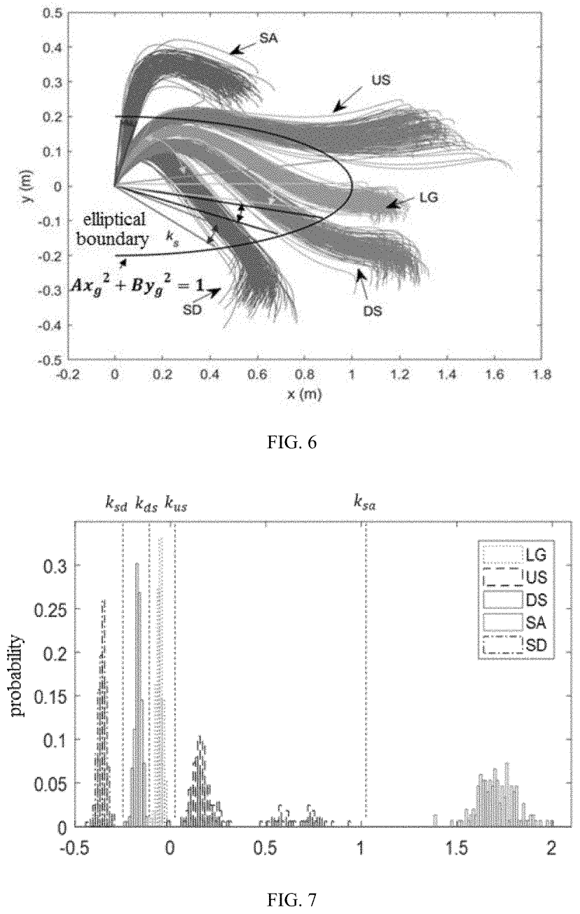

FIG. 6 shows a schematic diagram of an elliptical boundary condition for triggering motion pattern recognition decision for human lower limb according to an embodiment of the present application, wherein a motion pattern decision will be triggered when the obtained absolute motion trajectory to ground passes through the elliptical boundary condition. The elliptical boundary condition may be expressed as the following equation (2):

AX_{g}²+By_{g}²=1 (2)

Where A and B are constants, and x_{g} and y_{g} are coordinates of the obtained absolute motion trajectory in the x-axis direction and the y-axis direction.

As shown in FIG. 6, the absolute motion trajectories to ground of the lower limb extremity end of human body can be clearly distinguished from each other in different motion patterns. For example, FIG. 6 shows the absolute motion trajectory to ground of the lower limb extremity end under the motion patterns of the flat ground walking LG, the upslope US, the downslope DS, the upstairs SA, and the downstairs SD. In addition, an elliptical boundary condition for triggering a pattern detection decision is also shown in FIG. 6. When the obtained absolute motion trajectory to ground of the lower limb extremity end passes through the elliptical boundary trajectory, the slope of ground kₛ(t) will be obtained by the following equation (3) based on the displacement x_{g}(t) in the forward direction and the displacement y_{g}(t) in the direction vertical to the ground of lower limb extremity end.

$${k}_{s} ( t ) = \frac{{y}_{g} ( t )}{{x}_{g} ( t )} \qquad ( 3 )$$

In addition to the above elliptical boundary conditions, the boundary conditions according to exemplary embodiments of the present application may further include:

(1) Boundary conditions such as circles, rectangles, and the like, i.e., pattern recognition is triggered when the above absolute motion trajectory to ground passes through boundary conditions such as circles, rectangles, or the like;

(2) A time threshold value that triggers pattern recognition at a predetermined point in time;

(3) A displacement threshold value of the limb extremity end in a forward direction or a direction vertical to the ground;

(4) An acceleration threshold or angular velocity threshold in the sensor coordinate system;

In addition to the pattern recognition triggering condition described above, a data window may be used to monitor the motion pattern in real time to ensure that the motion pattern is predicted before the next foot contacting the ground.

FIG. 7 shows a probability distribution map for the slope of ground obtained according to the above equation when the absolute motion trajectory to ground of the lower limb extremity end passes through the elliptical boundary condition shown in FIG. 6. In this embodiment, a simple threshold-based pattern classifier is used. For example, four thresholds, as shown in FIG. 7, may be provided to accurately distinguish the five common motion patterns of the human body, i.e., upslope, downslope, upstairs, downstairs, and flat ground walking. Different motion patterns of the human body can be distinguished by the following equation (4):

$$LM = \{ \begin{matrix}SA & {k}_{s} ( t ) > {k}_{sa} \\ US & {k}_{sa} > {k}_{s} ( t ) > {k}_{us} \\ LG & {k}_{us} > {k}_{s} ( t ) > {k}_{ds} \\ DS & {k}_{ds} > {k}_{s} ( t ) > {k}_{sd} \\ SD & {k}_{s} ( t ) > {k}_{sd}\end{matrix} \} \qquad ( 4 )$$

Where, kₛ(t) is the slope of ground obtained at time t; kₛₐ is a threshold value for distinguishing the upstairs motion pattern from the upslope motion pattern; kᵤₛ is a threshold value for distinguishing the upslope motion pattern from the flat ground walking motion pattern; k_{ds} is a threshold value for distinguishing the flat ground walking motion pattern from the downslope motion pattern; and k_{sd} is a threshold value for distinguishing the downslope motion pattern from the downstairs motion pattern.

It should be noted that in order to realize motion pattern recognition, common classification patterns, for example, any one of a linear discriminant analyzer, a secondary discriminant analyzer, a support vector machine, or a neural network, may also be used to process one or more of the above obtained conversion matrix, the absolute displacement to ground, the absolute velocity to ground, and the absolute acceleration to ground.

In an exemplary embodiment, in order to detect turning activity of human body, the rotation angle or angular velocity of the human head, upper torso, arm, thigh, lower leg, foot or other parts of the body relative to the initial sagittal or coronal plane of the human body during turning may also be measured. The rotation angle or angular velocity can be measured by using the inertial measurement unit installed at a corresponding part of the human body, or the conversion matrix can be obtained by detecting the angular velocity and the acceleration in the sensor coordinate system, thereby obtaining the rotation angle or angular velocity.

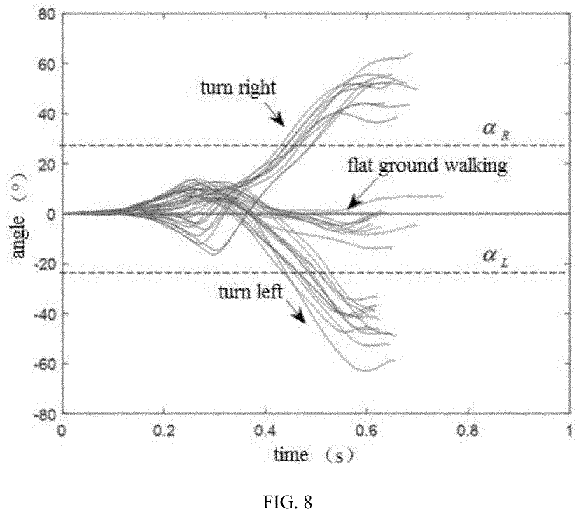

FIG. 8 shows a schematic diagram for recognizing a turning motion of a human body based on a rotation angle of the lower limb extremity end of human body relative to an initial sagittal plane of the human body according to an embodiment of the present application. Referring to FIG. 8, it shows that the turning motion of the human body is determined or detected based on the rotation angle of the human foot relative to the initial sagittal plane of the human body. As shown in the following equation (5), when the rotation angle is greater than a predetermined threshold value α_{R}, the human body performs a turn right TR motion; when the rotation angle is less than a predetermined threshold value α_{L}, the human body performs a turn left TL motion:

$$TA = \{ \begin{matrix}turn right & α > {α}_{R} \\ turn left & α < {α}_{L}\end{matrix} \qquad ( 5 )$$

Thresholds α_{R} and α_{L} may be determined by training the pattern recognizer or the classifier, and the turning motion of the human body may be recognized using the trained pattern recognizer or classifier.

Further, although not shown, in the exemplary embodiment, the turning motion of the human body may also be recognized based on the angular velocity of the lower limb extremity end of human body relative to the initial sagittal plane or the initial coronal plane of the human body obtained by detection.

In one or more embodiments, aspects of the present patent document may be directed to, may include, or may be implemented on one or more information handling systems (or computing systems). An information handling system/computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data. For example, a computing system may be or may include a personal computer (e.g., laptop), tablet computer, mobile device (e.g., personal digital assistant (PDA), smart phone, phablet, tablet, etc.), smart watch, server (e.g., blade server or rack server), a network storage device, camera, or any other suitable device and may vary in size, shape, performance, functionality, and price. The computing system may include random access memory (RAM), one or more processing resources such as a central processing unit (CPU) or hardware or software control logic, read only memory (ROM), and/or other types of memory. Additional components of the computing system may include one or more disk drives, one or more network ports for communicating with external devices as well as various input and output (I/O) devices, such as a keyboard, mouse, stylus, touchscreen and/or video display. The computing system may also include one or more buses operable to transmit communications between the various hardware components.

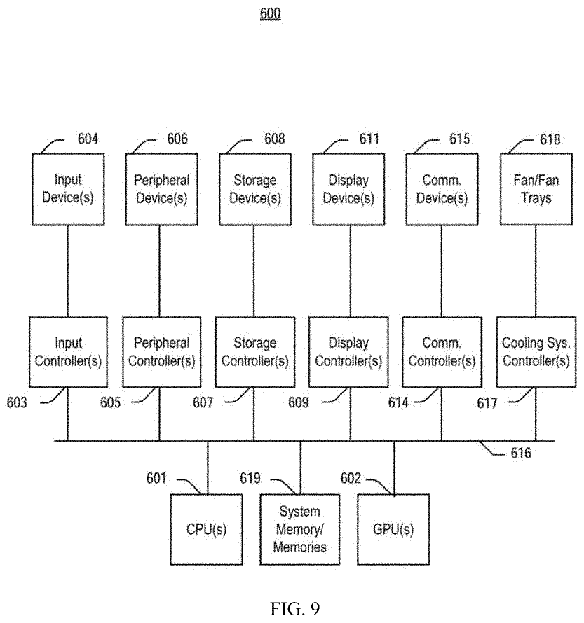

FIG. 9 depicts a simplified block diagram of an information handling system (or computing system) for the above method of recognizing the motion pattern of a limb, according to embodiments of the present disclosure.

It will be understood that the functionalities shown for system 600 may operate to support various embodiments of a computing system—although it shall be understood that a computing system may be differently configured and include different components, including having fewer or more components as depicted in FIG. 9.

As illustrated in FIG. 9, the computing system 600 includes one or more central processing units (CPU) 601 that provides computing resources and controls the computer. CPU 601 may be implemented with a microprocessor or the like, and may also include one or more graphics processing units (GPU) 602 and/or a floating-point coprocessor for mathematical computations. In one or more embodiments, one or more GPUs 602 may be incorporated within the display controller 609, such as part of a graphics card or cards. Thy system 600 may also include a system memory 619, which may comprise RAM, ROM, or both.

A number of controllers and peripheral devices may also be provided, as shown in FIG. 9. An input controller 603 represents an interface to various input device(s) 604, such as a keyboard, mouse, touchscreen, and/or stylus. The computing system 600 may also include a storage controller 607 for interfacing with one or more storage devices 608 each of which includes a storage medium such as magnetic tape or disk, or an optical medium that might be used to record programs of instructions for operating systems, utilities, and applications, which may include embodiments of programs that implement various aspects of the present disclosure. Storage device(s) 608 may also be used to store processed data or data to be processed in accordance with the disclosure. The system 600 may also include a display controller 609 for providing an interface to a display device 611, which may be a cathode ray tube (CRT) display, a thin film transistor (TFT) display, organic light-emitting diode, electroluminescent panel, plasma panel, or any other type of display. The computing system 600 may also include one or more peripheral controllers or interfaces 605 for one or more peripherals 606. Examples of peripherals may include one or more printers, scanners, input devices, output devices, sensors, and the like. A communications controller 614 may interface with one or more communication devices 615, which enables the system 600 to connect to remote devices through any of a variety of networks including the Internet, a cloud resource (e.g., an Ethernet cloud, a Fiber Channel over Ethernet (FCoE)/Data Center Bridging (DCB) cloud, etc.), a local area network (LAN), a wide area network (WAN), a storage area network (SAN) or through any suitable electromagnetic carrier signals including infrared signals. As shown in the depicted embodiment, the computing system 600 comprises one or more fans or fan trays 618 and a cooling subsystem controller or controllers 617 that monitors thermal temperature(s) of the system 600 (or components thereof) and operates the fans/fan trays 618 to help regulate the temperature.

In the illustrated system, all major system components may connect to a bus 616, which may represent more than one physical bus. However, various system components may or may not be in physical proximity to one another. For example, input data and/or output data may be remotely transmitted from one physical location to another. In addition, programs that implement various aspects of the disclosure may be accessed from a remote location (e.g., a server) over a network. Such data and/or programs may be conveyed through any of a variety of machine-readable medium including, for example: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, other non-volatile memory (NVM) devices (such as 3D XPoint-based devices), and ROM and RAM devices.

Aspects of the present disclosure may be encoded upon one or more non-transitory computer-readable media with instructions for one or more processors or processing units to cause steps to be performed. It shall be noted that the one or more non-transitory computer-readable media shall include volatile and/or non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, for example: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, other non-volatile memory (NVM) devices (such as 3D XPoint-based devices), and ROM and RAM devices. Examples of computer is code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into modules and/or sub-modules or combined together.

In the foregoing specification, embodiments of the disclosure have been described with reference to specific exemplary embodiments thereof. It will be evident that various modifications may be made thereto without departing from the broader spirit and scope of the disclosure as set forth in the following claims. The specification and drawings are, accordingly, to be regarded in an illustrative sense rather than a restrictive sense.

What is claimed is:

1. A method for recognizing a motion pattern of a limb, comprising:
collecting, by a sensor, motion data of a limb extremity end of a subject during a swing stage of the extremity end in different motion modes;
training a classifier or a pattern recognizer by inputting the collected motion data and corresponding limb motion patterns into the classifier or the pattern recognizer to train; and
recognizing the motion pattern of the limb by inputting the motion data of the limb, which is obtained in real time by the sensor, into the trained classifier or the trained pattern recognizer.

2. The method according to claim 1, wherein,
the limb comprises at least one of a lower limb, a lower limb prosthesis, a lower limb orthosis, or a lower limb exoskeleton of a human body, and
the motion pattern comprises at least one of upslope, downslope, upstairs, downstairs, walking on flat ground, and turning.

3. The method according to claim 2, wherein,
the motion data comprises one or more of an absolute motion trajectory to ground, an absolute velocity to ground, and an absolute acceleration to ground of the limb extremity end during the swing stage in the different motion modes.

4. The method according to claim 3, wherein, the sensor comprises an inertial measurement unit fixed to the limb extremity end, and
wherein the method further comprises:
obtaining one or more of the absolute motion trajectory to ground, the absolute velocity to ground, and the absolute acceleration to ground, through a coordinate transformation and an integration of angular velocity and acceleration data of the inertial measurement unit, which are obtained in a sensor coordinate system.

5. The method according to claim 4, further comprising:
resetting, in response to the human body being in a standing stage, a transformation matrix for the coordinate transformation, the absolute velocity to ground, and an absolute motion displacement to ground, to eliminate or reduce a cumulative drift or cumulative error of the inertial measurement unit.

6. The method according to claim 5, further comprising:
detecting the standing stage of the subject, by the inertial measurement unit fixed at the limb extremity end or a load cell mounted on a foot of the subject.

7. The method according to claim 3, wherein the collecting comprises:
extracting the absolute motion trajectory to ground of the limb extremity end in a sagittal plane, and
deriving terrain slopes corresponding to the different motion patterns from the absolute motion trajectory to ground in the sagittal plane to recognize the motion pattern being performed.

8. The method according to claim 4, further comprising:
triggering, based on a trigger boundary condition, the trained classifier or the trained pattern recognizer to recognize the motion pattern performed by the subject before a foot of the subject touching ground, wherein the motion pattern of the subject is recognized in response to the trigger boundary condition being satisfied.

9. The method according to claim 8, wherein, the trigger boundary condition comprises an elliptical boundary condition, a circular boundary condition, or a rectangular boundary condition, wherein the motion pattern of the subject is recognized in response to the absolute motion trajectory to ground of the limb extremity end passing through the trigger boundary condition.

10. The method according to claim 8, wherein, the trigger boundary condition comprises one or more of a time threshold trigger, an absolute displacement to ground trigger in a forward direction or a direction vertical to ground, an absolute velocity to ground trigger, or an absolute acceleration to ground trigger.

11. The method according to claim 8, wherein, the trigger boundary condition comprises: one or more of the angular velocity or acceleration signals of the inertial measurement unit in the sensor coordinate system satisfy a preset trigger condition.

12. The method according to claim 4, further comprising:
detecting, based on a time window, the motion pattern of the subject in real time to recognize the motion pattern performed by the subject before a foot of the subject touches the ground,
wherein the motion pattern of the subject is recognized in response to one or more of the absolute velocity to ground, the absolute acceleration to ground or the absolute motion trajectory to ground matching, within the time window, a corresponding data of a particular motion pattern.

13. The method according to claim 4, wherein the collecting comprises:
calculating a rotation angle or angular velocity of the limb extremity end relative to an initial sagittal plane or an initial coronal plane of the subject to recognize turning activity of the subject.

14. The method according to claim 13, further comprising:
obtaining the rotation angle or angular velocity of the limb extremity end relative to the initial sagittal plane or the initial coronal plane of the subject by converting output data of the inertial measurement unit fixed to the limb extremity end, or
recognizing the turning activity of the subject by detecting the rotation angle or angular velocity of other parts of the body of the subject relative to the initial sagittal plane or the initial coronal plane of the subject.

15. The method according to claim 14, wherein the other parts of the body comprise one or more of head, upper torso, aims, lower thighs, lower legs, and feet.

16. The method according to claim 1, wherein, the classifier or the pattern recognizer comprises a linear discriminant analyzer, a quadratic discriminant analyzer, a support vector machine, or a neural network.

17. The method according to claim 3, wherein
the sensor includes an inertial measurement unit-combined laser displacement sensor mounted on lower legs, thighs, waists, or head of the subject, and
wherein the inertial measurement unit-combined laser displacement sensor is configured to,
measure one or more of the absolute motion trajectory to ground, the absolute velocity to ground, or the acceleration to ground; or
measure topographic characteristics in the different motion patterns.

18. The method according to claim 3, wherein
the sensor includes an inertial measurement unit-combined depth camera mounted to lower legs, thighs, waists, or head of the subject, and
wherein the inertial measurement unit-combined depth camera is configured to,
measure one or more of the absolute motion trajectory to ground, the absolute velocity to ground, or the acceleration to ground, or
measure topographic characteristics in the different motion patterns.

19. The method according to claim 3, wherein, the sensor comprises an infrared capture system mounted in an ambient environment of the subject, and an infrared capture marker point is mounted at the limb extremity end of the subject, and
wherein the mothed further comprises:
analyzing one or more of the absolute motion trajectory to ground, the absolute velocity to ground, and the absolute acceleration to ground of the infrared capture marker point to recognize the motion pattern of the subject.

20. The method according to claim 3, further comprising:
recognizing one or more different motion patterns by combining one or more of the absolute motion trajectory to ground, the absolute velocity to ground, and the absolute acceleration to ground with a foot pressure distribution of the subject, a rotation angle of a lower limb knee joint or ankle joint, an electromyographic signal or an electroencephalographic signal (EEG) of the subject.

21. The method according to claim 3, further comprising:
recognizing one or more different motion patterns by combining one or more of the absolute motion trajectory to ground, the absolute velocity to ground, and the absolute acceleration to ground with an angular velocity or an acceleration in a sensor coordinate system measured by an inertial measurement unit fixed at the limb extremity end.

22. A non-transitory machine-readable medium storing instructions executable by a processor to perform:
collect, by a sensor, motion data of a limb extremity end of a subject during a swing stage of the extremity end in different motion modes;
train a classifier or a pattern recognizer by inputting the collected motion data and corresponding limb motion patterns into the classifier or the pattern recognizer to train; and
recognize the motion pattern of the limb by inputting the motion data of the limb, which is obtained in real time by the sensor, into the trained classifier or the trained pattern recognizer.

23. A data processing system comprising:
a processor; and
a memory coupled to the processor to store instructions executable by the processor to perform:
collect, by a sensor, motion data of a limb extremity end of a subject during a swing stage of the extremity end in different motion modes;
train a classifier or a pattern recognizer by inputting the collected motion data and corresponding limb motion patterns into the classifier or the pattern recognizer to train; and
recognize the motion pattern of the limb by inputting the motion data of the limb, which is obtained in real time by the sensor, into the trained classifier or the trained pattern recognizer.
